(19) United States
(12) Patent Application Publication
(10) Pub. No.: US 2011/0091946 A1
(43) Pub. Date: Apr. 21, 2011

(54) Enzymatic Process for Obtaining a Fatty Ester

(76) Inventors: Rosa Maria Teixeira Tage Biaggio, Sao Jose dos Campos (BR); André Luis da Silva Novaes, Sao Paulo (BR)

(21) Appl. No.: 12/990,354

(22) PCT Filed: Apr. 30, 2009
(86) PCT No.: PCT/BR09/00113

§ 371 Date: Jan. 6, 2011

(30) Foreign Application Priority Data
Apr. 30, 2008 (FR) 0852949

(51) Int. Cl. C12P 7/64

(52) U.S. Cl. 435/134

(57) ABSTRACT

The aim of the present invention is to obtain fatty esters from fatty acids derived from vegetable oils and butters, through an enzymatic process, so that said esters can act as emollients, emulsifiers and co-emulsifiers with differentiated performance, having high spreadability and slidability compared to the prior-art products. More specifically, the present invention relates to an enzymatic process for obtaining a fatty ester, comprising: a) obtaining a fatty acid through the enzymatic hydrolysis (saponification) of vegetable oils and butters selected from cupuagu butter, sapucainha butter, ucu\acute{υ}ba butter, murumuru butter, palm olein, pataua oil, tucuma oil, inaja oil, passion fruit oil, pequi oil and nasturtium oil; b) reacting the fatty acid with a fatty alcohol selected from capric alcohol, caprylic alcohol, isoamylic alcohol, lauric alcohol, myristic alcohol, lorol (70:30 mixture of lauric alcohol and myristic alcohol), cetyl alcohol, stearyl alcohol, cetostearyl alcohol (70:30 mixture of cetyl alcohol and stearyl alcohol), sorbitol, sorbitan, glycerin, polyglycerin, other polyols and mixture thereof, in the presence of an enzyme, in at least one reactor, at a temperature of from 40 to 80 QC; c) stirring the reaction solution; d) removing moisture from the reaction medium; e) vacuum filtering said solution to obtain the fatty ester. The fatty esters obtained through the process of the present invention can be used in the preparation of cosmetic compositions.

### INTRODUCTION

The aim of the present invention is to obtain fatty esters from fatty acids derived from vegetable oils and butters, through an enzymatic process, so that said esters can act as emollients, emulsifiers and co-emulsifiers with differentiated performance, having high spreadability and slidability compared to the prior-art products.

More specifically, the present invention relates to an enzymatic process for obtaining a fatty ester, comprising:

a) obtaining a fatty acid through the enzymatic hydrolysis (saponification) of vegetable oils and butters selected from cupuaçu (Theobroma grandiflorum) butter, sapucainha (Carpotroche Brasiliensis) butter, ucuúba (Virola sebifera) butter, murumuru (Astrocaryum murumuru) butter, palm olein, patau ucuá (Oenocarpus bataua) oil, tucumä (Astrocaryum aculeatum) oil, inajá (Maximiliana Maripa) oil, passion fruit oil, pequi (Caryocar brasiliense) oil and nasturtium oil;

b) reacting the fatty acid with a fatty alcohol selected from capric alcohol, caprylic alcohol, isoamylic alcohol, lauric alcohol, myristic alcohol, lorol (70:30 mixture of lauric alcohol and myristic alcohol), cetyl alcohol, steparyl alcohol, cetostearyl alcohol (70:30 mixture of cetyl alcohol and stearyl alcohol), sorbitol, sorbitan, glycerin, polyglycerin, other polyols and mixture thereof, in the presence of an enzyme, in at least one reactor, at a temperature of from 40 to 80° C.;

c) stirring the reaction solution;

d) removing moisture from the reaction medium;

e) vacuum filtering said solution to obtain the fatty ester.

The fatty esters obtained through the process of the present invention can be used in the preparation of cosmetic compositions.

### STATE OF THE ART

Document U.S. Pat. No. 6,933,139 B2 discloses a method for the enzymatic splitting of fatty acids and glycerol obtained from oils and fats by using lipases being added to a mixture containing an oil or fat and water. The splitting reaction is performed only up to a splitting degree at which slowing-down of the splitting reaction is still below a preset value using discontinuously operated loop reactors. The fatty acids to be obtained are separated from the reaction mixture that is only partially split, by first separating an aqueous glycerol-containing phase from a partially split organic phase containing split fatty acids, in a self-cleaning centrifugal separator. Afterwards, the fatty acids are separated from the partially split organic phase and the residue of the organic phase freed from the free fatty acids is fed back into the splitting process.

Therefore, said document discloses neither the step of removing the humidity of the reaction medium, in particular by means of an inline flash tank, to distill the water generated during the esterification process nor the use of an enzymatic recirculation reactor. Additionally, the document does not disclose the use of immobilized enzymes either, and the lipases used in the document and in the present invention are different, since they have different specificities. Finally, the starting material of the present invention is different from the starting triglycerides of said document.

Document U.S. Pat. No. 5,219,733 discloses a process for reacting a component selected from the group consisting of sterols and branched aliphatic primary or secondary alcohols having 14 to 32 carbon atoms, and a component selected from the group consisting of fatty acids and fatty acid esters in contact with an enzyme selected from the group consisting of lipase and cholesterol esterase or with the selected enzyme in an immobilized form, in a system selected from the group consisting of an aqueous medium and water-containing organic solvent to prepare a fatty acid ester of the initial component.

Said document does not disclose the use of oils such as the cupuaçu butter and the sapucainha butter. Additionally, said document does not disclose the use of lipases derived from the microorganisms Candida Antarctica, Rhizomucor miehei and Thermomyces lanuginosus either. Therefore, the enzymes used in the present invention are not anticipated by the document above. Finally, the document does not disclose the use of a completely organic reaction medium, but rather of an aqueous medium containing an organic solvent to solubilize one of the phases.

Document US 2005/0014237 A1 discloses a method to improve the productivity in an enzymatic method for making esterified, transesterified or interesterified products. Specifically, it discloses a method that can improve the productivity of enzymatic transesterification or esterification by deodorization alone, or by deodorization and purification of the initial substrate to extend the useful life of the enzymes.

The document describes a method that is different from that of the present invention and does not disclose the use of fatty acids selected from capric alcohol, caprylic alcohol, isoamylic alcohol, lauric alcohol, myristic alcohol, lorol (70:30 mixture of lauric alcohol and myristic alcohol), cetyl alcohol, stearyl alcohol, cetostearyl alcohol (70:30 mixture of cetyl alcohol and stearyl alcohol), sorbitol, sorbitan, glycerin, polyglycerin, other polyols and mixture thereof.

### OBJECT OF THE INVENTION

The objective of the present invention is to obtain fatty esters from fatty acids derived from vegetable oils and butters, through an enzymatic process, so that said esters can act as emollients, emulsifiers and co-emulsifiers with differentiated performance, having high spreadability and slidability compared to standard formulae.

The fatty esters obtained through the process of the present invention can be used in the preparation of cosmetic compositions.

### DESCRIPTION OF DRAWINGS

FIG. 1 illustrates a glass-lined multipurpose stirred reactor, for use in the process of the present invention when a single reactor is used, for example, in a batch process.

FIG. 2 schematically illustrates the esterification process of the present invention when 4 reactors in series are used, in a semi-continuous or continuous process.

FIG. 3 contains data about the pH of the formulations of cupuaçu fatty ester with myristol (Formula 3) and cupuaçu ester with myristol/Cupuaçu ester with sorbitol (Formula 6).

FIG. 4 contains data about the viscosity of the formulations of cupuaçu fatty ester with myristol (Formula 3) and cupuaçu ester with myristol/cupuaçu ester with sorbitol (Formula 6).

FIG. 5 shows the stability evaluation of the cupuaçu fatty ester with myristol, at 5° C., 37° C., 45° C., light and dark conditions (room temperature) for 7, 14, 30, 60 and 90 days, based on the Acidity Index (AI) and Water Content (%).

FIG. 6 shows the results of “in vitro” tests obtained by comparing the absorbance values between the standard formulation, the formulation in which the solubilization system was substituted and the film-forming system was substituted.

FIG. 7 shows the results of “in vivo” tests for Sun Protection Factor in dry conditions.

FIG. 8 shows the results of water resistance tests of the formulations after 2 hours.

FIG. 9 contains the results obtained in the barrier booster tests performed by comparing the formulas with the cupuaçu ester with myristol to evaluate the performance as a skin barrier booster. Four formulations were tested: placebo formula; formula with 1% myristyl cupuçuate; formula with 3% myristyl cupuçuate and formula with 6% myristyl cupuçuate, thus obtaining response curves in relation to hydration.

FIG. 10 shows the sensorial profile of the formulations comprising the esters obtained from the process of the present invention.

### DESCRIPTION OF THE INVENTION

The present invention relates to an enzymatic process for obtaining a fatty ester, comprising:

a) obtaining a fatty acid through the enzymatic hydrolysis (saponification) of vegetable oils and butters selected from cupuaçu butter, sapucainha butter, ucuúba butter, murumuru butter, palm olein, patauá oil, tucumä oil, inajá oil, passion fruit oil, pequi oil and nasturtium oil;

b) reacting the fatty acid with a fatty alcohol selected from capric alcohol, caprylic alcohol, isoamylic alcohol, lauric alcohol, myristic alcohol, lorol (70:30 mixture of lauric alcohol and myristic alcohol), cetyl alcohol, stearyl alcohol, cetostearyl alcohol (70:30 mixture of cetyl alcohol and stearyl alcohol), sorbitol, sorbitan, glycerin, polyglycerin, other polyols and mixture thereof, in the presence of an enzyme, in at least one reactor, at a temperature of from 40 to 80° C.;

c) stirring the reaction solution;

d) removing moisture from the reaction medium;

e) vacuum filtering said solution to obtain the fatty ester.

### DETAILED DESCRIPTION OF THE INVENTION

The present invention relates to an enzymatic process for obtaining a fatty ester, comprising a step of melting the fatty acid. Then the fatty acid is reacted with a fatty alcohol, in the presence of an enzyme, in at least one reactor, at a temperature of from 40 to 80° C. The reaction solution is stirred and the humidity is removed from the reaction medium. Finally, said solution is vacuum filtered to obtain the fatty ester.

The fatty acids used as raw-material for the process of the present invention are fatty acids obtained through a process of enzymatic hydrolysis (saponification) of butters and oils selected from cupuaçu butter, sapucainha butter, ucuúba butter, murumuru butter, palm olein, patauá oil, tucumä\ddot{} oil, inajá oil, passion fruit oil, pequi oil and nasturtium oil. Preferably, they are selected from cupuaçu butter, sapucainha butter and palm olein.

The saponification process of the butter or oil can be carried out by adding a base at a temperature of from 80 to 90° C. Preferably, the base used is KOH or NaOH.

After this step, H₂SO₄ is added, which results in the obtainment of said fatty acids, which may be washed until they reach a neutral pH. The enzymatic hydrolysis process of the butter or oil can be carried out by using 1.5% of the enzyme blend relative to the butter or oil, in three additions of 0.5% and with the addition of the same amount of water by weight, corresponding to the amount of butter used. The additions were made every 12 hours, totaling over 48 hours.

The fatty acids obtained through the process described above are then used as raw-material in the enzymatic esterification process of the present invention.

The enzymatic esterification is carried out by reacting said fatty acids obtained from the vegetable oils and butters with fatty alcohols.

Said fatty alcohol can be selected from capric alcohol, caprylic alcohol, isoamylic alcohol, lauric alcohol, myristic alcohol, lorol (70:30 mixture of lauric alcohol and myristic alcohol), cetyl alcohol, stearyl alcohol, cetostearyl alcohol (70:30 mixture of cetyl alcohol and stearyl alcohol), sorbitol, sorbitan, glycerin, polyglycerin, other polyols and mixture thereof. Preferably, lorol, mystiric alcohol, cetostearyl alcohol or sorbitan are used.

The reaction between the fatty acid and the fatty alcohol in the presence of an enzyme is carried out in at least one enzymatic reactor. Said at least one enzymatic reactor is preferably a fixed bed enzymatic reactor. Preferably, the enzyme to be used is selected from lipases, particularly the Novozymes® 435 (lipase B), Lipozyme® RM 1M and Lipozyme® TL IM lipases, derived from the microorganisms Candida antarctica, Rhizo-mucor miehei and Thermomyces lanuginosus, respectively. The aforementioned lipases are commercialized by Novozymes. Preferably, the lipase used is Novozymes® 435. In accordance with one embodiment of the invention the enzyme used is a blend consisting of CALB and Lypozyme TL 100 L at the ratio of 1:9 or 9:1 added at a temperature of from 50 to 60° C., followed the addition of water.

Preferably, the enzyme selected is immobilized. The lipases Novozymes® 435 (lipase B) and Lipozyme® RM IM are thermoresistant enzymes, and the support used is of polymeric origin. In the case of the lipase Lipozyme® TL 1M, the support used is silica.

The reactor used in the process of the present invention is selected from a single reactor, two reactors in series or four reactors in series. The process can be a batch, semi-continuous or continuous process.

Particularly, the process of the present invention is carried out in stoichiometric/molar scale, the preferred fatty acid to enzyme ratio being 1:1.1 in moles. Preferably, the enzyme represents about 2 to 10% by weight of the total weight of the fatty acid.

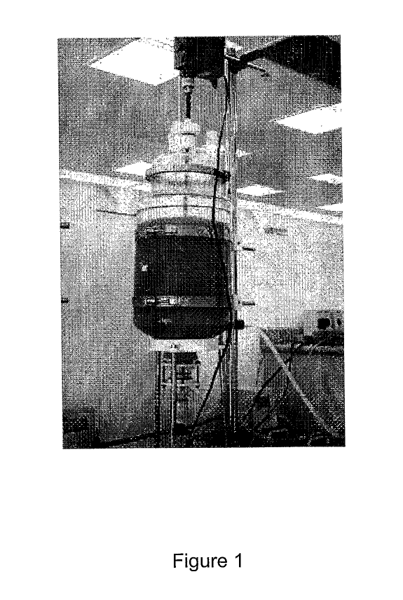

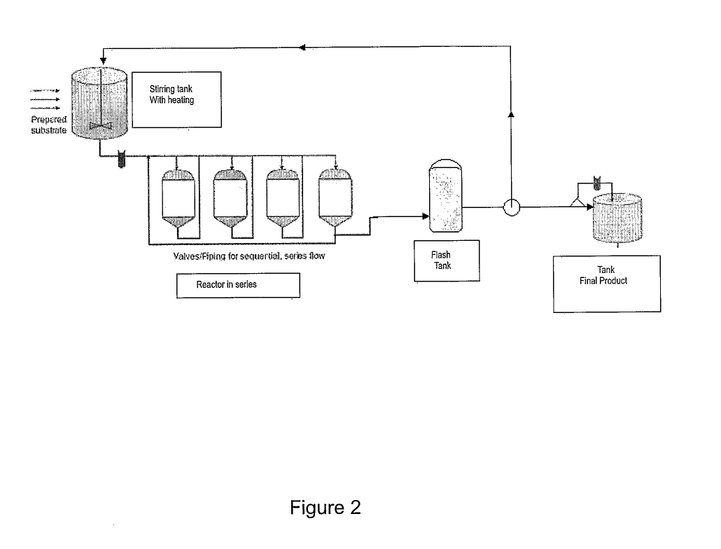

In case a single reactor is used, for example, for use in a batch process, said reactor can be a multipurpose stirred reactor, preferably a glass-lined reactor (FIG. 1) or a 316 stainless steel reactor. The reactor has a distillation system connected thereto, so that the humidity generated during the esterification process can be removed and the reaction equilibrium can be shifted towards the formation of the product, that is, of the fatty ester, thus speeding up the reaction.

When a single reactor is used, the fatty acid is previously molten. The reactor should be lined and the circulating liquid can be water or thermal oil. The reactor is heated to a temperature of from 55 to 75° C., preferably from 60 to 70° C. When the temperature of the lining reaches about 50° C., the molten fatty acid and the alcohol are added, followed by the addition of the enzyme. The reaction medium is then stirred at about from 276 to 300 rpm, preferably at 276 rpm. Finally, the product obtained is vacuum filtered through a filter with porosity between 0.01 and 0.1 mm, which filter can be made of stainless steel or any other type of mesh.

In case two or more reactors in series are used, preferably four reactors in series, they can have an internal “basket” containing said enzyme. The use of reactors in series is particularly useful in the semi-continuous and continuous processes.

Additionally, a stirred, and optionally heated, feed tank can be connected to the system to homogenize the substrates (fatty acid and fatty alcohol). The substrates circulate through the reactors, through a loop, and may return or not to the feed tank, according to the esterification result. The substrate must circulate for a period of about 5 hours for the product to be obtained, making the process semi-continuous.

After it has passed through the last reactor, a flash tank is optionally connected. The purpose of the flash tank is to remove the excess humidity generated in the esterification process, thus shifting the reaction equilibrium towards the formation of the fatty ester.

When two or more reactors, preferably four reactors in series, are used, the fatty acid and/or the fatty alcohol can be previously molten. The two reagents are then weighed and transferred to the feed tank and mixed. The bath is then heated to about 70° C. and the recirculation is activated. At the end of the process, the product is vacuum discharged.

Typically, the reaction time is of 5 to 72 hours and the reaction is carried out at atmospheric pressure. However, the presence of vacuum in the system significantly decreases the reaction time, in addition to preventing the oxidation of the unsaturated fatty chains, such as the oleic, linoleic and linoleis acids. Additionally, the presence of vacuum increases the contact surface between the reagents, favoring the formation of the product.

Both in the case where only one reactor is used and in the case where two or more reactors in series are used, the process can be followed and controlled through the acidity index.

The process can be controlled through the acidity index (mgKOH/g), by the AOCS method, in which the product is titrated with a titrated solution of 0.1N aqueous KOH. The results of the acidity indexes are shown in Table 1.

TABLE 1

| | Acidity Index |
|---|---|
| Product | (mgKOH/g) |
| Process with a single reactor | |
| Sapucainha Ester with Lorol (Example 1) | 5.25 |
| Sapucainha Ester with Myristol (Example 2) | 3.80 |
| Cupuaçu Ester with Sorbitol (Example 3) | 43.0 |
| Cupuaçu Ester with Sorbitan | 35.7 |
| Palm Olein Ester with Sorbitol | 31.10 |
| Palm Olein Ester with Sorbitan | 32.21 |
| Process in enzymatic reactor | |
| Cupuaçu Ester with Lorol (Example 4) | 4.60 |
| Cupuaçu Ester with Myristol (Example 5) | 15.00 |
| Palm Olein Ester with Cetostearyl alcohol | 12.20 |
| (Example 6) | |

The esterification conversion through the process described above provides a esterification conversion of 95 to 100% and a process yield of 85 to 98%.

### EXAMPLES

The examples below are preferred and illustrative embodiments of the esterification process of the present invention and should not be interpreted as limitations thereof. In this sense, it is to be understood that the scope of the present invention embraces other possible variations, being limited solely by the appended claims, including the possible equivalents therein.

### Example 1

### Esterification of Sapucainha Fatty Acid with Lorol

Multifunctional Gas-lined Reactor (batch process)
Raw materials

| | | |
|---|---|---|
| Sapucainha Fatty Acid (I60001) | 2300.0 | g (1.0 mol) |
| Lorol CD (Batch: HN4J298560) | 1820.0 | g (1.1 mol) |
| Novozymes ® 435 Enzyme | 184.0 | g (8% in relation |
| (Batch: LC200206) | | to fatty acid) |

### Process

1) The fatty acid was previously melted.

2) The bath was activated at 60° C.

3) When the bath temperature reached 50° C., fatty acid and lorol CD were added.

4) The temperature was increased to 60° C.

5) The enzyme was added to the reactor.

6) The stirrer was turned on at 276 rpm.

7) After the specification was met, the product was discharged by vacuum filtration.

### Example 2

### Esterification of Sapucainha Fatty Acid with Myristol

Multifunctional Gas-lined Reactor (batch process)
Raw materials

| | | |
|---|---|---|
| Sapucainha Fatty Acid (Batch: 02/14/07) | 1900.0 | g (1 mol) |
| Myristol (Batch: HN6A264205) | 1594.0 | g (1.1 mol) |
| Novozymes ® 435 Enzyme | 152.0 | g (8% in relation |
| (Batch: LC200206) | | to fatty acid) |

### Process

1) The fatty acid was previously melted.

2) The bath was activated at 60° C.

3) When the bath temperature reached 50° C., fatty acid and lorol CD were added.

4) The temperature was increased to 60° C.

5) The enzyme was added to the reactor.

6) The stirrer was turned on at 276 rpm.

7) After the specification was met, the product was discharged by vacuum filtration.

### Example 3

### Esterification of Cupuaçu Fatty Acid with Sorbitol

Multifunctional Gas-lined Reactor (batch process)
Raw materials

| | | |
|---|---|---|
| Cupuaçu Fatty Acid (Batch 001/06) | 2580.0 | g (1.0 mol) |
| Sorbitol 70% (Batch: H70019) | 2400.0 | g (1.0 mol) |
| Novozymes ® 435 Enzyme | 200.0 | g (8% in relation |
| (Batch: LC200212) | | to the fatty acid). |

### Process

1) The fatty acid was previously melted.

2) The bath was activated at 70° C.

3) When the bath temperature reached 50° C., fatty acid and 1290.0 g of Sorbitol 70% were added.

4) The enzyme was added to the reactor.

5) The stirrer was turned on at 300 rpm.

6) On the 2^{nd} day, 1110.0 g of Sorbitol 30% was added.

7) The product was discharged by vacuum filtration.

### Example 4

### Esterification of Cupuaçu Fatty Acid with Lorol CD

Stainless Steel Reactors in series (semi-continuous process)
Bath temperature=70° C.

### Pressure=0.3 bar

Raw materials

| | | |
|---|---|---|
| Cupuaçu Fatty Acid (Batch: 001/06) | 20.0 | Kg (1 mol) |
| Lorol CD (Batch: HN6B034965) | 150 | Kg (1.1 mol) |
| Novozymes ® 435 Enzyme | 5.0 | Kg (8% in relation |
| | | to the fatty acid) |

1 Kg (Batch: LC200206)
4 Kg (Batch: LC200212) - reused enzyme

### Process

1) The sapucainha fatty acid was previously melted in the feed tank.

2) The reactor was heated for the product to reach 70° C.

3) Recirculation was turned on.

4) After 12 hours, the product was vacuum discharged.

### Example 5

### Esterification of Cupuaçu Fatty Acid with Myristol (Lanette 14)

Stainless Steel Reactors in series (semi-continuous process)
Bath temperature=70²C

### Pressure=0.20 bar

Raw materials

| | | |
|---|---|---|
| Cupuaçu Fatty Acid (Batch: 001/06) | 201 | Kg (1 mol) |
| Myristol (Batch: HN6A264205) | 188 | Kg (1.1 mol) |
| Novozymes ® 435 Enzyme | 5.0 | Kg (8% in relation |
| | | to the fatty acid) |

1 Kg (Batch: LC200206)
4 Kg (Batch: LC200212) - reused enzyme

### Process

1. The cupuaçu fatty acid and myristol was previously melted.

2. The two reagents are then weighed and transferred to the feed tank and mixed.

3. The bath was heated for the product to reach 70° C.

4. Recirculation was turned on.

5. After 12 hours, the product was vacuum discharged.

### Example 6

### Esterification of Palm Olein Fatty Acid with Cetostearyl Alcohol

Stainless Steel Reactors in series (semi-continuous process)
Bath temperature=70° C.

### Pressure=0.25 bar

Raw materials

| | | |
|---|---|---|
| Palm Olein Fatty Acid (Batch: 001/06) | 25.0 | Kg (1 mol) |
| Cetostearyl Alcohol (Batch: H40016) | 24.3 | Kg (1.1 mol) |
| Novozymes ® 435 Enzyme | 5.0 | Kg (8% in relation |
| | | to the fatty acid) |

1 Kg (Batch: LC200206)
4 Kg (Batch: LC200212) - reused enzyme

### Process

1) The palm olein fatty acid and the cetostearyl alcohol were previously melted.

2) The two reagents are then weighed and transferred to the feed tank and mixed.

3) The bath was heated for the product to reach 70° C.

4) Recirculation was turned on.

5) After 12 hours, the product was vacuum discharged.

The esters obtained from the process of the present invention were tested in terms of safety, efficacy and use as sun filter solubilizers. Furthermore, a sensorial analysis was performed of the esters obtained from the process of the present invention. This data is indicated below.

### Safety Tests

The safety of the esters obtained from the process of the present invention was tested according to conventional methods for assessing cytotoxicity, PC5 and complete irritation. The results obtained are indicated in Table 2.

TABLE 2

| Test | Result |
|---|---|
| Cytotoxicity | Non-toxic |
| PC5 | Non-irritating |
| Complete irritation | Non-allergenic |

### Efficacy Tests

Esters obtained from the process of the present invention were assessed as emulsifiers in galenic forms. By assessing the stability of the formulations in comparison with emulsifiers commonly available in the market, it was possible to approve the performance of the esters as emulsifiers.

Thus, from a known base O/W formula as described in Table 3, 13 formulations were prepared, as indicated in Table 4, wherein the cetearyl olivate/sorbitan olivate (Oliven 1000) of said base formula was substituted with sorbitan olivate (Oliven 900) and fatty esters and sorbitol esters obtained from fatty acids of cupuaçu, sapucainha and palm olein.

TABLE 3

| | | CONCENTRATION |
|---|---|---|
| PHASE | COMPONENT | (%) |
| 1 | DEMINERALIZED WATER | 60.74 |
| 1 | DISODIUM EDTA | 0.10 |
| 1 | BIDISTILLED GLYCERIN | 8.00 |
| | BXR VEGETABLE | |
| 2 | ALKYL ACRYLATE TR-1 | 0.20 |
| 2 | XANTHAN GUM | 0.50 |
| 3 | DICAPRYL ETHER | 2.00 |
| 3 | CETYL LACTATE | 1.00 |
| 3 | BHT | 0.10 |
| 3 | CETEARYL OLIVATE, | 3.00 |
| | SORBITAN OLIVATE | |
| 3 | GLYCERYL STEARATE | 0.50 |
| 3 | DICAPRYLYL CARBONATE | 2.00 |
| 4 | CYCLOMETHICONE D5/D6 VS7158 | 5.00 |
| 4 | CYCLOMETHICONE AND | 6.00 |
| | DIMETHICONE CROSSPOLYMER | |
| 5 | BIOSACCHARIDE GUM-1 | 7.00 |
| 6 | NYLON 12 | 2.00 |
| 7 | IODOPROPYNYL | 0.10 |
| | BUTYLCARBAMATE | |
| 7 | PHENOXYETHANOL F | 0.60 |
| 8 | DEMINERALIZED WATER | 1.00 |
| 8 | TRIETHANOLAMINE | 1.60 |

TABLE 4

| FORMULA | EMULSIFER |
|---|---|
| 1 | Cetostearyl olivate, sorbitan olivate |
| 2 | Cupuaçu ester with sorbitol |
| 3 | Cupuaçu ester with myristol |
| 4 | Cupuaçu ester with lorol |
| 5 | Sorbitan olivate (Oliven 900) |
| 6 | Cupuaçu ester with sorbitol + |
| | Cupuaçu ester with myristol (30:70) |
| 7 | Cupuaçu ester with sorbitol + |
| | Cupuaçu ester with lorol (30:70) |
| 8 | Sapucainha ester with myristol |
| 9 | Sapucainha ester with lorol |
| 10 | Sapucainha ester with sorbitol |
| 11 | Sapucainha ester with sorbitol + |
| | Sapucainha ester with myristol (30:70) |
| 12 | Sapucainha ester with sorbitol + |
| | Sapucainha ester with lorol (30:70) |
| 13 | Palm olein ester with cetostearyl alcohol |

The stability of formulations 1 to 13 was analyzed at 5° C., 45° C., 50° C. and dark conditions (room temperature) for 7, 14, 30, 60 and 90 days. The results obtained are indicated in Table 5.

TABLE 5

| FORMULA | EMULSIFER | STABILITY |
|---|---|---|
| 1 | Cetostearyl olivate, sorbitan olivate | OK |
| 2 | Cupuaçu ester with sorbitol | Separated after |
| | | 60 days (45° C. |
| | | and 50° C.) |
| 3 | Cupuaçu ester with myristol | OK |
| 4 | Cupuaçu ester with lorol | OK |
| 5 | Sorbitan olivate (Oliven 900) | Separated after |
| | | centrifugation |
| 6 | Cupuaçu ester with sorbitol + | OK |
| | Cupuaçu ester with myristol (30:70) | |
| 7 | Cupuaçu ester with sorbitol + | OK |
| | Cupuaçu ester with lorol (30:70) | |
| 8 | Sapucainha ester with myristol | OK |
| 9 | Sapucainha ester with lorol | OK |
| 10 | Sapucainha ester with sorbitol | Separated after |
| | | centrifugation |
| 11 | Sapucainha ester with sorbitol + | OK |
| | Sapucainha ester with myristol (30:70) | |
| 12 | Sapucainha ester with sorbitol + | OK |
| | Sapucainha ester with lorol (30:70) | |
| 13 | Palm olein ester with cetostearyl | OK |
| | alcohol | |

Analyzing the results, it is concluded that isolated fatty esters can act as emulsifiers without the need of sorbitol esters, as in the case of Oliven 1000 in the base O/W formula used. Oliven 900 is more substituted and therefore more non-polar and has a lower HLB, being more appropriate for W/O formulations. The sorbitol ester produced through the process of the present invention is less substituted, having a higher HLB and, therefore, better performance in O/W formulation, which is reflected in its performance, keeping the emulsion stability for up to 60 days at 45° C. and 50° C. Oliven 900 separated after centrifugation, immediately after the preparation of the formulation.

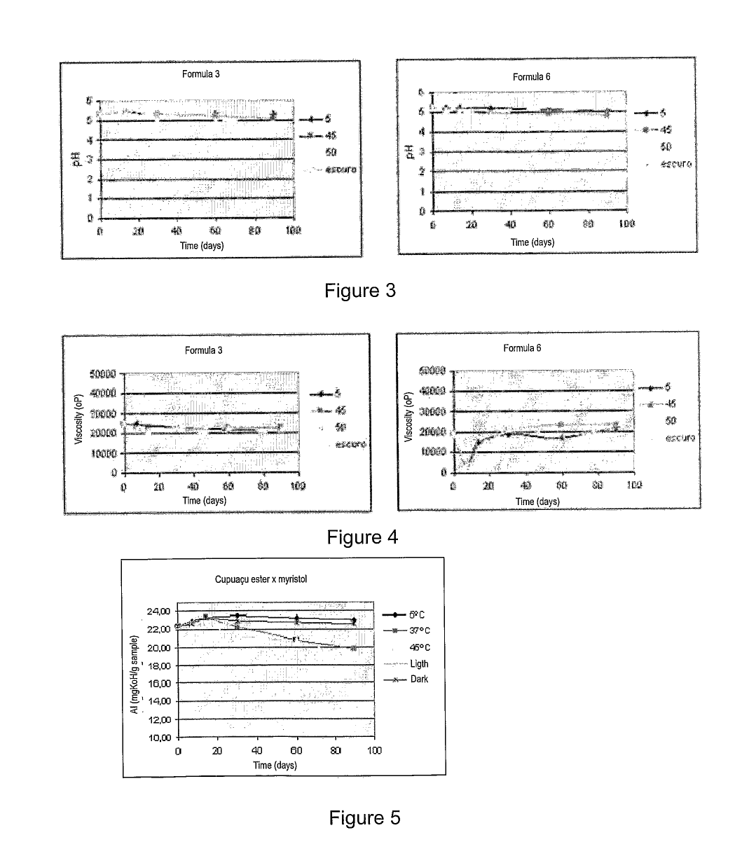

The stability data (pH and viscosity) of the formulations of cupuaçu fatty ester with myristol (Formula 3) and cupuaçu ester with myristol/cupuaçu ester with sorbitol (Formula 6) is indicated in FIGS. 3 and 4.

The stability of the cupuaçu fatty ester with myristol was assessed at 5° C., 37° C., 45° C., light and dark conditions (room temperature) for 7, 14, 30, 60 and 90 days. Said stability was assessed based on the Acidity Index (AI) and Water Content (%). The results are represented in FIG. 5:

### Use Test of the Esters Obtained by the Process of the Present Invention as Sun Filter Solubilizinq Agents

The esters obtained from the process of the present invention were also tested as physical and chemical sun filter solubilizing agents in the standard formulation for SPF 30 described in Table 6 below:

TABLE 6

| | | CONCENTRATION | AMOUNT |
|---|---|---|---|
| PHASE | COMPONENT | (%) | (G) |
| 01 | Demineralized water | 55.8260 | 55.8260 |
| 01 | Disodium EDTA | 0.1000 | 0.1000 |
| 02 | Carbomer 980 | 0.4000 | 0.4000 |
| 03 | Xanthan Gum | 0.2000 | 0.2000 |
| 04 | Bidistilled Glycerin BXR Vegetable | 5.000 | 5.000 |
| 04 | Bisethylhexyloxyphenolmethoxy | 1.5000 | 1.5000 |
| | phenyl triazine | | |
| 04 | Ethylhexyl Methoxycinnamate | 7.5000 | 7.5000 |
| 04 | Benzophenone-3 | 5.0000 | 5.0000 |
| 04 | Dicaprylyl Carbonate | 3.5000 | 3.5000 |
| 04 | Stearyl dimethicone | 1.0000 | 1.0000 |
| 04 | Dimethicone Trimethylsiloxysilicate | 1.5000 | 1.5000 |
| 04 | C₁₂₋₁₅ Alkyl Benzoate | 6.0000 | 6.0000 |
| 04 | BHT | 0.0500 | 0.0500 |
| 04 | Dimethicone Copolyol ethyl ether | 0.5000 | 0.5000 |
| 05 | Titanium dioxide and simethicone | 2.0000 | 2.0000 |
| 06 | Potassium cetyl phosphate | 2.0000 | 2.0000 |
| 07 | Demineralized water | 3.0000 | 3.0000 |
| 07 | Triethanolamine | 0.5000 | 0.5000 |
| 08 | Cyclomethicone D5 and dimethiconol | 1.0000 | 1.0000 |
| 08 | Cyclomethicone D5/D6 VS7158 | 2.0000 | 2.0000 |
| 09 | Lycopene | 0.0020 | 0.0020 |
| 09 | Tocopheryl acetate (Vitamin E) | 0.2000 | 0.2000 |
| 10 | Demineralized water | 0.0200 | 0.0200 |
| 10 | Coffee extract (Coffea robusta) | 0.0020 | 0.0020 |
| 11 | Iodopropynyl Butylcarbamate | 0.2000 | 0.2000 |
| 11 | Phenoxyethanol F | 1.0000 | 1.0000 |

In the base formula above, filter solubilizing emollients, such as C12-C15 alkyl benzoate and dicapryl carbonate were substituted with the fatty esters obtained from the process of the present invention, and the film-forming silicones, which promote resistance to the formulation, were substituted with Sapucainha and Ucuúba butters. The prepared formulations are as described in Table 7 below:

TABLE 7
SPF 30 EMULSION

| | EMOLLIENTS | | FILM-FORMER |
|---|---|---|---|
| BDP | 6% | 3.5% | 3% |
| 134.2218.5 | Alkyl benzoate | Dicapryl | Dimethicone and |
| | | | trimethyl: 1.5% |
| | | | Dimethicone |
| | | | steraryl: 1.0% |
| | | | Copolyol ether |
| | | | (PEG-8): 0.5% |
| 134.2218.9 | Sapucainha ester | Cupuaçu ester | Sapucainha butter |
| | w/lorol | w/myristol | |
| 134.2218.10 | Alkyl benzoate | Cupuaçu ester | Sapucainha butter |
| | | w/lorol | |
| 134.2218.11 | Sapucainha ester | Cupuaçu ester | Ucuúba butter |
| | w/lorol | w/myristol | |
| 134.2218.12 | Dicapryl | Cupuaçu ester | Sapucainha butter |
| | | w/myristol | |
| 134.2218.13 | Sapucainha ester | Babaçu mono- | Sapucainha butter |
| | w/lorol | glyceride | |
| 134.2218.14 | Cupuaçu ester | Babaçu mono- | Sapucainha butter |
| | w/lorol | glyceride | |

BDP 134.2218.5 - SPF 30 Photoequilibrium Formula according to the base formula of Table 6)
BDP 134.2218.11 - Solubilization and film-forming system substitution
BDP 134.2218.9 - Solubilization and film-forming system substitution
BDP 421.10417.1 - Solubilization system substitution
BDP 421.10417.2 - Film-forming system substitution

The main changes with regard to the standard formula according to Table 6 are indicated in Table 8 below:

TABLE 8

| | BDP 134.2218.11 | BDP 134.2218.9 | BDP 421.10417.1 | BDP 421.10417.2 |
|---|---|---|---|---|
| | Solubilization | Solubilization | Solubilization | Film-forming |
| | and Film-forming | and Film-forming | system | system |
| | system substitution | system substitution | substitution | substitution |
| Removed | Dicapryl carbonate | Dicapryl carbonate | Dicapryl carbonate | Dimethicone stearyl |
| | Dimethicone stearyl | Dimethicone stearyl | Alkyl benzoate | Dimethicone |
| | Dimethicone | Dimethicone | | Copolyol ether |
| | Copolyol ether | Copolyol ether | | |
| | Alkyl benzoate | Alkyl benzoate | | |
| Included | Ucuúba butter | Sapucainha butter | Cupuaçu | Ucuúba butter |
| | Cupuaçu | Cupuaçu | fatty ester | |
| | fatty ester | fatty ester | Sapucainha | |
| | Sapucainha | Sapucainha | fatty ester | |
| | fatty ester | fatty ester | | |

### Stability

The stability of the formulations above was evaluated by electronic microscopy to check the crystallization of the sun filters. The formulations were stable and there was no separation of the emulsion or filter crystallization.

### Sun Protection Factor (SPF) “In Vitro” Tests

The “in vitro” tests determined that in the formulations in which only the esters (solubilization system) were substituted and the silicones maintained intact, there was no change in the SPF in relation to the standard SPF 30 formulation. In the formulations were the silicones (film-formers) and the esters were substituted simultaneously, there was a reduction in the SPF in relation to the standard SPF 30 formulation.

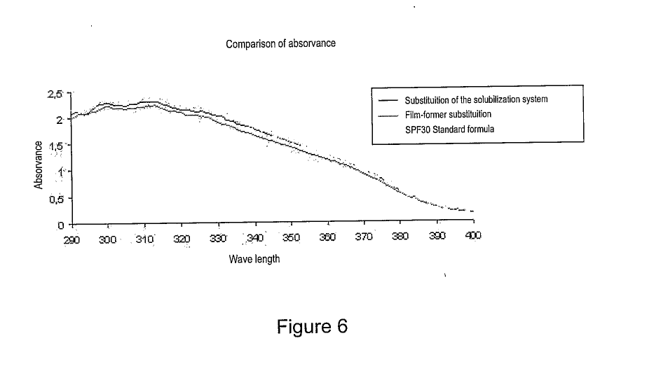

The “in vitro” test was carried out by comparing the absorbance values of the standard formulations and those in which the sun filter solubilizing emollients were substituted according to Table 7. The result of this test can be seen in FIG. 6 and in Tables 9 and 10:

TABLE 9
SPF 30 EMULSION

| | EMOLLIENTS | | FILM-FORMER |
|---|---|---|---|
| BDP | 6% | 3.5% | 3% |
| 134.2218.5 | Alkyl benzoate | Dicapryl | Dimethicone and |
| | | | trimethyl: 1.5% |
| | | | Dimethicone |
| | | | steraryl: 1.0% |
| | | | Copolyol ether |
| | | | (PEG-8): 0.5% |
| 134.2218.9 | Sapucainha ester | Cupuaçu ester | Sapucainha butter |
| | w/lorol | w/myristol | |
| 421.10417.2 | Alkyl benzoate | Dicapryl | Ucuúba butter |
| 134.2218.11 | Sapucainha ester | Cupuaçu ester | Ucuúba butter |
| | w/lorol | w/myristol | |
| 421.10417.1 | Sapucainha ester | Cupuaçu ester | Silicones |
| | w/lorol | w/myristol | |

TABLE 10

| Sample | Description | SPF |
|---|---|---|
| 134.2218.5 | Standard Formula | 78.69 |
| 421.10417.1 | Solubilization system substitution | 74.69 |
| 421.10417.2 | Film-forming system substitution | 67.09 |
| 134.2218.9 | Substitution of both systems | 64.7 |
| 134.2218.11 | Substitution of both systems | 65.8 |

### SPF “In Vivo” Tests

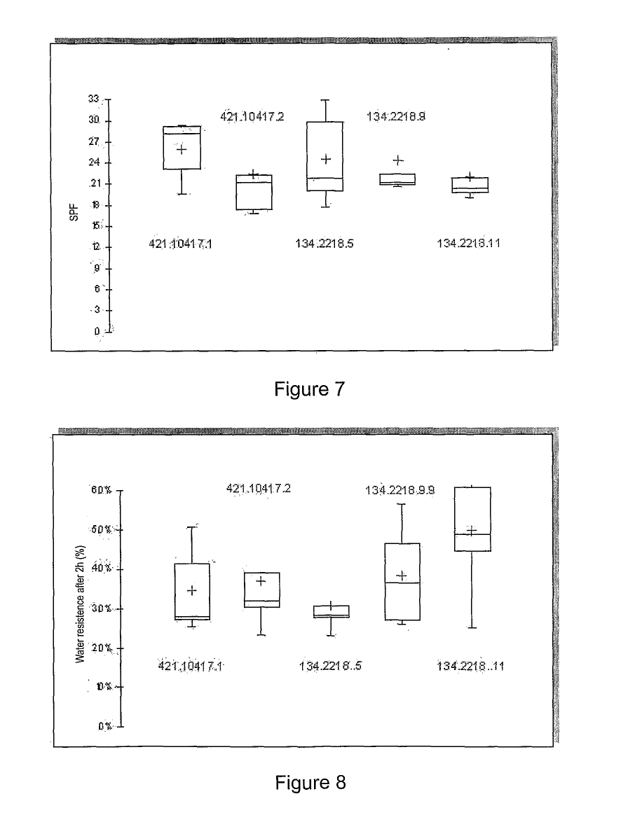

The “in vivo” tests for assessing SPF were carried out according to the most accepted methodologies for assessing the SPF of a protector, which considers biological responses associated with the protection against UVB radiation. The clinical assessments were made according to international protocols (FDA, COLIPA, JCIA, etc). The results relating to SPF and water resistance are indicated in FIGS. 7 and 8.

### Barrier Boost Test

Tests were carried out with formulas containing with the cupuaçu ester with myristol to assess the performance as skin barrier booster. Four formulations were tested: placebo formula; formula with 1% myristyl cupuçuate; formula with 3% myristyl cupuçuate and formula with 6% myristyl cupuçuate, thus obtaining response curves related with hydration. The skin barrier strengthening potential of the cupuaçu ester with myristol was assessed through the use of cosmetic products after mechanical insult through the tape-stripping removal procedure and subsequent readings of transepidermal water loss (TEWL) in the skin of voluntary individuals during 15 days.

Table 11 shows the formula with 3% myristyl cupuçuate. The other preparations have analogous formulas, in which only the concentration of myristyl cupuçuate is altered.

TABLE 11

| | | CONCENTRATION | AMOUNT |
|---|---|---|---|
| PHASE | COMPONENT | (%) | (G) |
| 01 | Demineralized water | 84.4000 | 84.4000 |
| 01 | Alkyl acrylate TR-1 | 0.6500 | 0.6500 |
| 01 | Disodium EDTA | 0.1000 | 0.1000 |
| 02 | Xanthan Gum | 0.2500 | 0.2500 |
| 02 | Glyceryl monostearate and PEG | 1.0000 | 1.0000 |
| | 100 Stearate | | |
| 02 | Dicaprylyl Carbonate | 3.5000 | 3.5000 |
| 02 | C₁₂₋₁₅ Alkyl Benzoate | 2.5000 | 2.5000 |
| 02 | Cupuaçu fatty ester with myristol | 3.0000 | 3.0000 |
| 02 | Isononyl Isononanoate | 1.0000 | 1.0000 |
| 02 | Cetyl Lactate | 2.0000 | 2.0000 |
| 03 | Iodopropynyl Butylcarbamate | 0.2000 | 0.2000 |
| 03 | Phenoxyethanol F | 1.0000 | 1.0000 |
| 04 | Triethanolamine | 0.4000 | 0.4000 |

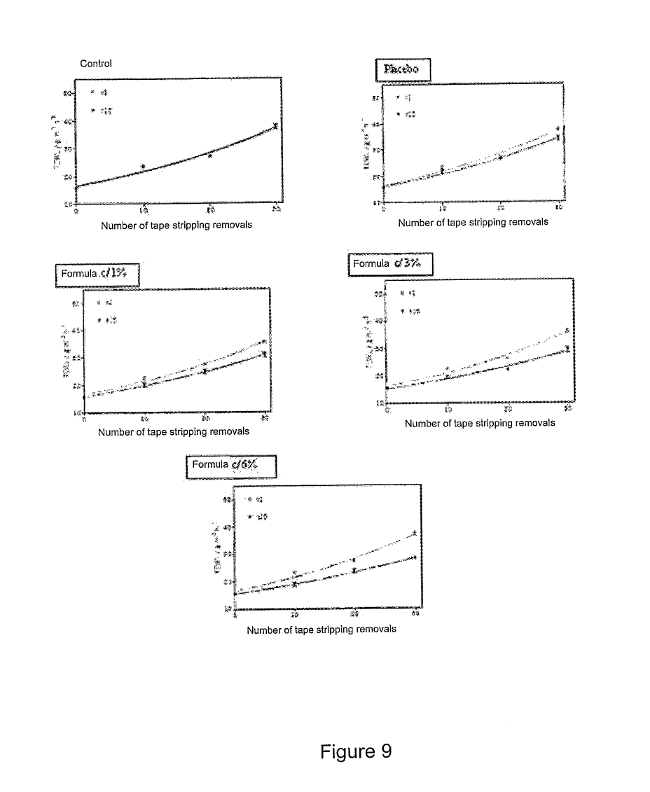

The results of the abovementioned tests showed that hydration increased statistically as concentration increased, indicating a positive response to the concentration curve and the fatty ester performance as a skin barrier strengthener. The barrier booster test results are shown in FIG. 9.

### Benefit-Related Tests

The esters prepared according to the process of the present invention were assessed from basic O/W galenic formulations according to Table 12 below:

TABLE 12

| | | CONCENTRATION |
|---|---|---|
| PHASE | COMPONENT | (%) |
| 1 | DEMINERALIZED WATER | 60.74 |
| 1 | DISODIUM EDTA | 0.10 |
| 1 | BIDISTILLED GLYCERIN BXR VEGETABLE | 8.00 |
| 2 | ALKYL ACRYLATE TR-1 | 0.20 |
| 2 | XANTHAN GUM | 0.50 |
| 3 | DICAPRYL ETHER | 2.00 |
| 3 | CETYL LACTATE | 1.00 |
| 3 | BHT | 0.10 |
| 3 | CETEARYL OLIVATE, SORBITAN OLIVATE | 3.00 |
| 3 | GLYCERYL STEARATE | 0.50 |
| 3 | DICAPRYLYL CARBONATE | 2.00 |
| 4 | CYCLOMETHICONE D5/D6 VS7158 | 5.00 |
| 4 | CYCLOMETHICONE AND DIMETHICONE | 6.00 |
| | CROSSPOLYMER | |
| 5 | BIOSACCHARIDE GUM-1 | 7.00 |
| 6 | NYLON 12 | 2.00 |
| 7 | IODOPROPYNYL BUTYLCARBAMATE | 0.10 |
| 7 | PHENOXYETHANOL F | 0.60 |
| 8 | DEMINERALIZED WATER | 1.00 |
| 8 | TRIETHANOLAMINE | 1.60 |

Formulas were prepared in which the emulsifiers were substituted according to Table 13 below; in addition, separately a formula was prepared in which all silicones (11.0%) were removed and to which 3% of cupuaçu ester with myristol was added (formula 421.7019.15), totaling 14% of the ester.

TABLE 13

| FORMULA | EMULSIFER | STABILITY |
|---|---|---|
| 421.7019.1 | Cetostearyl olivate, sorbitan olivate | OK |
| 421.7019.3 | Cupuaçu ester with myristol | OK |
| 421.7019.4 | Cupuaçu ester with lorol | OK |
| 421.7019.6 | Cupuaçu ester with sorbitol + | OK |
| | Cupuaçu ester with myristol (30:70) | |
| 421.7019.7 | Cupuaçu ester with sorbitol + | OK |
| | Cupuaçu ester with lorol (30:70) | |
| 421.7019.8 | Sapucainha ester with myristol | OK |
| 421.7019.9 | Sapucainha ester with lorol | OK |
| 421.7019.13 | Palm olein ester with cetostearyl alcohol | OK |
| 421.7019.14 | Olein ester with sorbitol + | OK |
| | olein ester with cetostearyl alcohol (30:70) | |
| 421.7019.15 | Cupuaçu ester with myristol | OK |

The aspects relating to the benefits were classified and analyzed according to the definitions below:
Absorption point (Pabs): Number of rotations needed for the product to start being absorbed by the skin;
Spreadability (Esp): Easiness to spread the product on the skin;
Slidability (DesI): Easiness to slide/move the finger on the skin;
Immediate skin brightness (Bri im): Light intensity reflected on the skin immediately after the product is spread;
Residual skin brightness (Bri res): Light intensity reflected on the skin two minutes after the product is spread;
Stickiness (Peg): Intensity with which the finger adheres to the skin;
Immediate oleosity (Ole im): Oil sensation on the skin during and after the product is spread;
Residual oleosity (Ole res): Oil sensation on the skin 2 minutes after the product is spread;
Immediate fat film (F gord im): Fat sensation forming a film on the skin immediately after the product is spread;
Residual fat film (F gord res): Fat sensation forming a film on the skin 2 minutes after the product is spread;
Velvety film (Favel): Peach skin sensation;
White residue: Formation of a white film on the skin.

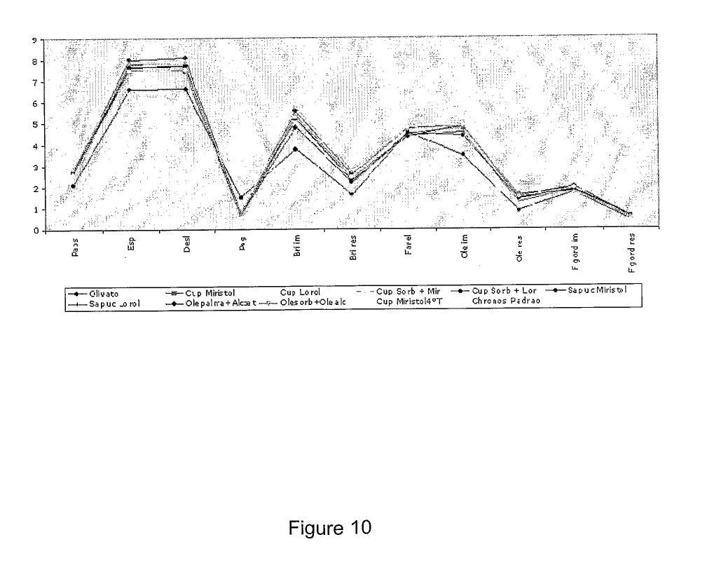

From the definitions above, it was possible to determine a sensorial profile of the formulations prepared. Said profiles are represented in Table 14 below and in FIG. 10.

TABLE 14

| Products | Properties | Applications | Differential |
|---|---|---|---|
| Sapucainha Ester | Good spreadability | High Spreadability emollient | High spreadability |
| with Lorol | Slidability | Chemical filter solubilizer | Dry touch |
| | Low stickiness | Co-emulsifier | Liquid |
| Sapucainha Ester | Spreadability | Emollient | Dry touch, solid |
| with Myristol | Slidability | Chemical Filter solubilizer | waxy with low melting |
| | Low stickiness | Co-emulsifier | point |
| Cupuaçu ester | Spreadability | High Spreadability emollient | High brightness |
| with lorol | Slidability | Chemical filter solubilizer | Spreadability |
| | Skin brightness | Co-emulsifier | |
| | Low stickiness | Skin luminosity | |
| Cupuaçu ester | Velvety film | Skin barrier booster | Velvety film |
| with myristol | Spreadability | Emollient | Brightness |
| | Slidability | Chemical filter solubilizer | |
| | Low stickiness | Co-emulsifier | |
| Palm Olein ester | Spreadability | Emollient | Low cost |
| with cetostearyl | Slidability | Co-emulsifier | |
| alcohol | Low stickiness | | |
| Sorbitol ester with | O/W emulsifier | O/W emulsifier | O/W emulsifier |
| cupuaçu | | | |
| Sorbitol ester with | O/W emulsifier | O/W emulsifier | Low cost |
| palm olein | | | |

1. An enzymatic process for obtaining a fatty ester, characterized by comprising:
a) obtaining a fatty acid through the enzymatic hydrolysis (saponification) of vegetable oils and butters selected from the group consisting of cupuagu butter, sapucainha butter, ucuuba butter, murumuru butter, palm olein, pataud oil, tucuma oil, inaja oil, passion fruit oil, pequi oil and nasturtium oil;
b) reacting the fatty acid with a fatty alcohol selected from capric alcohol, caprylic alcohol, isoamylic alcohol, lauric alcohol, myristic alcohol, lorol (70:30 mixture of lauric alcohol and myristic alcohol), cetyl alcohol, stearyl alcohol, cetostearyl alcohol (70:30 mixture of cetyl alcohol and stearyl alcohol), sorbitol, sorbitan, glycerin, polyglycerin, other polyols and mixture thereof, in the presence of an enzyme, in at least one reactor, at a temperature of from 40 to 80° C.;
c) stirring the reaction solution;
d) removing moisture from the reaction medium;
e) vacuum filtering said solution to obtain the fatty ester.

2. The process according to claim 1, characterized in that the butters and oils are selected from the group consisting of cupuagu butter, sapucainha butter and palm olein.

3. The process according to claim 1, characterized in that the fatty alcohol is selected from the group consisting of lorol, myristic alcohol, cetostearyl alcohol, sorbitan and sorbitol.

4. The process according to claim 1, characterized in that the enzymatic hydrolysis is carried out by the addition of an enzyme blend to the vegetable oil or butter, at a temperature of from 50 to 60° C., followed by the addition of water.

5. The process according to claim 4, characterized in that the enzyme blend consists of CALB and Lypozyme TL 100 L at the ratio of 1:9 or 9:1.

6. The process according to claim 1, characterized by comprising the additional step of melting the fatty acid before step (b) of the reaction.

7. The process according to claim 1, characterized by comprising the additional step of melting the fatty alcohol before step (b) of the reaction.

8. The process according to claim 1, characterized in that in step (b) the enzyme is a lipase.

9. The process according to claim 8, characterized in that the lipase is selected from the group consisting of Novozymes® 435, Lipozyme® RM IM and Lipozyme® TL IM.

10. The process according to claim 9, characterized in that the lipase is Novozymes® 435.

11. The process according to claim 1, characterized in that the enzyme represents about 8% by weight of the total weight of fatty acid.

12. The process according to claim 1, characterized in that at least one reactor is selected from a single reactor, two reactors in series or four reactors in series.

13. The process according to claim 12, characterized in that at least one reactor are four reactors in series.

14. The process according to claim 1, characterized in that the stirring step (c) is carried out at from 276 to 300 rpm.

15. The process according to claim 1, characterized in that the fatty acid and the fatty alcohol are prehomogenized in a stirred feed tank before the step (b) of the reaction.

16. The process according to claim 15, characterized in that the feed tank is heated.

17. The process according to claim 15, characterized in that, after the at least one reactor, there is a step of recirculating the reaction solution to the feed tank.

18. The process according to claim 1, characterized in that the step (d) of removing the moisture of the reaction medium is carried out by distillation or by a flash tank placed after at least one reactor.

19. The process according to claim 1, characterized in that at least one reactor is a fixed bed reactor.

20. The process according to claim 1, characterized in that the reaction time is from 5 to 72 hours.

21. The process according to claim 20, characterized in that the time is 8 hours, under vacuum.

22. The process according to claim 1, characterized in that in step (b) the reaction temperature is of from 50 to 75° C., at atmospheric pressure.

23. The process according to claim 22, characterized in that the reaction temperature is of from 60 to 70° C., at atmospheric pressure.

24. The process according to claim 1, characterized by comprising a step of process control.

25. The process according to claim 24, characterized in that the control is performed through a method selected from thin layer chromatography and acidity index.
